SEQUENCE LISTING (15 sequences)

SEQ ID NO: 1 - DNA, 36 bp, Unknown
  oligonucleotide
gagtgttatgggtagctcgagtgaagtgggaatatc

SEQ ID NO: 2 - DNA, 36 bp, Unknown
  oligonucleotide
gatattcccacttcactcgagctacccataacactc

SEQ ID NO: 3 - DNA, 36 bp, unknown
  oligonucleotide
gagtgttatgggtagcccgggtgaagtgggaatatc

SEQ ID NO: 4 - DNA, 36 bp, unknown
  oligonucleotide
gatattcccacttcacccgggctacccataacactc

SEQ ID NO: 5 - DNA, 36 bp, unknown
  oligonucleotide
gagtgttatgggtagctcgggtgaagtgggaatatc

SEQ ID NO: 6 - DNA, 36 bp, unknown
  oligonucleotide
gatattcccacttcacccgagctacccataacactc

SEQ ID NO: 7 - DNA, 36 bp, unknown
  oligonucleotide
gagtgttatgggtagcataactgaagtgggaatatc

SEQ ID NO: 8 - DNA, 36 bp, unknown
  oligonucleotide
gatattcccacttcagttatgctacccataacactc

SEQ ID NO: 9 - DNA, 42 bp, unknown
  oligonucleotide
gctgaagatcagttgggtgctcgagtgggttacatcgaactg

SEQ ID NO: 10 - DNA, 42 bp, unknown
  oligonucleotide
cagttcgatgtaacccactcgagcacccaactgatcttcagc

SEQ ID NO: 11 - DNA, 67 bp, unknown
  linker
ggatccgagctcgaattcgcggccgcacaccaccaccaccaccactagactagtggccaggccggcc

SEQ ID NO: 12 - DNA, 48 bp, unknown
  primer
agtcggatccgagctcttaaaggaacacgaaaatgagtcacccagatc

SEQ ID NO: 13 - DNA, 37 bp, unknown
  primer
agtcgaattcgtaaatctttgtcccatgttccattac

SEQ ID NO: 14 - DNA, 35 bp, unknown
  primer
gtaattattgcaagatatagacaagtaccttggat

SEQ ID NO: 15 - DNA, 35 bp, unknown
  primer
tacttgtctatatcttgcaataattacaggattca

(19) United States
(12) Patent Application Publication
(10) Pub. No.: US 2005/0003420 A1
(43) Pub. Date: Jan. 6, 2005

(54) Recycled mutagenesis of restriction endonuclease toward enhanced catalytic activity

(76) Inventors: Shuang-yong Xu, Lexington, MA (US); Zhenyu Zhu, Beverly, MA (US); Paul D. Riggs, Hooksett, NH (US); Pei-chung Hsieh, Topsfield, MA (US)

Correspondence Address: NEW ENGLAND BIOLABS, INC., 32 TOZER ROAD, BEVERLY, MA, 01915, US

(21) Appl. No.: 10/874,527

(22) Filed: Jun. 23, 2004

Related U.S. Application Data
(60) Provisional application No. 60/484,256, filed on Jul. 1, 2003.

(51) Int. Cl. C12Q 1/68; C07H 21/04; C12P 21/04; C12N 9/22

(52) U.S. Cl. 435/6; 435/69.1; 435/199; 435/320.1; 435/325; 536/23.2

(57) ABSTRACT

A method is described for increasing the activity of restriction endonuclease mutants that have altered binding or cleavage activities. Restriction endonuclease variants can carry one or more amino acid substitutions that change substrate specificity and at the same time decrease the enzyme catalytic activity. A method is described for isolating derivatives of the endonuclease variants by subjecting them to additional rounds of mutagenesis and screening in a dinD::lacZ indicator strain, such that second-site mutations within the nucleotide coding sequence of the endonuclease are obtained that increased the enzyme specific activity.

### CROSS REFERENCE

This application is a non-provisional application claiming priority of U.S. provisional application No. 60/484,256 filed Jul. 1, 2003, which is hereby incorporated by reference in its entirety.

### BACKGROUND OF THE INVENTION

Restriction endonucleases are both indispensable tools in manipulating genes and excellent enzyme models for studying sequence specific interactions with DNA. Over a dozen detailed structures of restriction endonucleases complexed with cognate or non-cognate DNA substrates have been described (Pingoud and Jeltsch, Nucl. Acids Res. 29:3705-3727 (2001)). The structural analysis of restriction endonucleases indicate that there are usually 15-20 hydrogen bonds between the amino acid side chains and the target sequence, plus a number of van der Waals contacts to the bases. In addition, multiple hydrogen bonds to the DNA backbone are observed within and outside the recognition sequence. The structure of the DNA target also undergoes significant bending and base stacking changes upon endonuclease binding. The extremely high specificity is critical to the survival of the host to prevent unnecessary double-stranded breaks on related sites within the host stranded breaks on related sites within the host chromosome. Such DNA damage could be detrimental to the host if the breaks are not resealed by DNA ligase or repaired by homologous recombination. Despite the requirement for extreme specificity, type II restriction endonucleases have evolved to recognize more than 200 unique sequences ranging from 3 to 8 bases (Roberts and Macelis, Nucl. Acids Res. 29:268-269 (2001). How do new specificities come into existence via natural evolutionary processes? Is it possible to engineer new restriction endonucleases from existing enzymes by directed evolution in the laboratory?

Several restriction enzymes have been subjected to structure-assisted engineering efforts. EcoRV (GATATC) has been studied extensively and high-resolution structures are available with cognate and non-cognate DNAs. By employing a semi-rational approach on a stretch of 22 amino acids, Pingoud et al. have isolated EcoRV variants which exhibit a 25-fold higher rate of cleaving EcoRV sites flanked by AT rather than GC base-pairs (Lanio, et al. J. Mol. Biol. 283:59-69 (1998)). Such additional sequence discrimination is possible because EcoRV contacts 3 additional base pairs on either side of its recognition sequence. Co-crystal structures of BamHI (GGATCC) complexed with cognate and non-cognate DNAs have been solved at high resolution. Site-directed and semi-random approaches to mutagenize BamHI restriction endonuclease resulted in a BamHI variant that preferentially cleaves a methylated BamHI site (Whitaker, et al. J. Mol. Biol. 285:1525-1536 (1999)). Random and site-directed amino acid substitutions of residues that make direct contacts with the BamHI recognition sequence did not generate BamHI mutants toward BglII site (AGATCT) or Sau3AI site (GATC) (Dorner, et. al. J. Mol. Biol. 285:1515-1523 (1999); Xu and Schildkraut J. Biol. Chem. 266:4425-4429 (1991)).

In structure-guided mutagenesis of EcoRI (GAATTC) and PvuII (CAGCTG) endonucleases, mutants with relaxed binding/cleavage specificities have been isolated (Nastri, et al. J. Biol. Chem. 272:25761-25767 (1997); Flores, et al. Gene 157:295-301 (1995)). In the PvuII variants, the amino acid changes that relaxed the DNA binding properties also diminished the DNA cleavage activity. By combining gene shuffling, PCR random mutagenesis, and methyltransferase selection, an Eco57I variant has been isolated that cleaves a degenerate sequence (CTGRAGN16/14, Janulaitis, et al. European Patent Application EP1179696A1).

Heitman and Model used an in vivo SOS induction assay to isolate relaxed specificity mutants of EcoRI from a randomly mutated library based on their ability to induce an SOS repair response in the presence of M.EcoRI protection. This in vivo selection implicated four residues important for specificity that were later observed at the protein-DNA interface in the revised EcoRI-DNA co-crystal structure (Heitman and Model EMBO J. 9:3369-3378 (1990)). A random mutagenesis approach employing a three-step genetic selection and screening has yielded BstYI (RGATCY) variants with increased specificity toward the BglII (AGATCT) DNA recognition sequence (Samuelson and Xu, J. Mol. Biol. 319:673-683 (2002), International Application No. PCT/US03/00542). The dinD::lacZ indicator strain employed in these studies has proven extremely useful for the direct cloning of thermophilic restriction enzyme genes in E. coli and for the isolation of mutants with relaxed or altered specificities.

BsoBI is a thermophilic isoschizomer of AvaI with the recognition sequence C/YCGRG (/cleavage site). The BsoBI co-crystal structure has been solved previously at high resolution (van der Woerd, et al. Structure 9:133-144 (2001)). The co-crystal structure of BsoBI revealed some unique features in DNA recognition and catalysis. There is a large tunnel in the BsoBI protein that completely encircles the DNA. A histidine residue (H253) was found near the catalytic site that is involved in base recognition (hydrogen bond to the inner guanine) and possibly also plays a role in catalysis as a general base to extract a proton from a nucleophilic water. Finally, a water-mediated hydrogen bond was found between D246 and the degenerate base adenine. It was proposed that by rotation of the water molecule, BsoBI endonuclease is capable of recognizing both adenine and guanine in the degenerate position. Interestingly, there are also two hydrogen bonds to the two bases immediately outside the recognition sequence, perhaps leading to preferences in the cleavage site with unique sequence context.

Structure-assisted engineering of endonuclease substrate specificity has achieved limited success in part due to the highly specific enzymes with multiple hydrogen bonds to the target sequence and employing a combination of direct and indirect read-out for DNA recognition. Furthermore, most protein residues involved in DNA recognition recognize more than one base or are also involved in catalysis. Thus, elimination of base-specific contacts has resulted in “dead” enzymes (null mutants without binding and cleavage activity), indicating the intimate coupling of base recognition and catalysis. Engineering of new enzyme specificity is further hindered by the requirement for a protective methyltransferase for host DNA protection when a new specificity arises.

U.S. Pat. No. 5,498,535 relates to a novel method for the direct cloning of nuclease genes such as restriction endonuclease genes in E. coli. In addition, there is provided a novel strain ER1992 (EcoKMR⁻, McrA⁻, McrBC⁻, Mrr⁻, dinD::lacZ) that facilitates application of the above method. This method has been successfully employed to clone a number of genes coding for nucleases including restriction endonuclease genes. This method for direct cloning of nuclease genes in E. coli used a strain containing a fusion of a DNA damage-inducing promoter and an indicator/reporter gene such as the dinD1::lacZ fusion. When ligated genomic DNA fragments and vector are introduced into an indicator strain such as dinD1::lacZ deficient in all restriction systems so far described and transformants plated on X-gal plates, one might find the nucleases-containing clone directly by picking blue colonies. When used to clone genes coding for a restriction endonuclease, unlike the methylase selection approach, it is not necessary that the methylase gene fully protect the host chromosome. In fact, the methylase gene may be absent altogether. This is particularly true for thermostable enzymes where the transformants are grown at lower temperatures, i.e., between about 30° C. to 37° C. At this lower temperature, thermostable restriction endonucleases are less active, and transformed host cells may survive with partial or even without protective methylation. The genes coding for the thermostable restriction enzymes BsoBI (5′CYCGRG3′), TaqI (5′TCGA3′), Tfi nuclease (non-specific), and Tth111I (5′GACNNNGTC3′) have been successfully cloned in E. coli by this method. The methylase selection method (U.S. Pat. No. 5,200,333) and the “endonuclease indicator method” can also be combined to clone restriction endonuclease genes. The gene coding for the restriction endonuclease ecoO109IR was cloned by combining the two methods.

Isolation of restriction endonuclease mutants with reduced activity or relaxed specificity had been described (Heitman and Model Gene. 103:1-9 (1991); Xu and Schildkraut J. Biol. Chem. 266:4425-4429 (1991); Waugh and Sauer J. Biol Chem. 269:12298-303 (1994)). It is extremely difficult to isolate mutants with complete change of specificity (or enhanced specificity) and high enzyme activity. Therefore, it is desirable to devise an in vivo/in vitro method to isolate endonuclease mutants with high specific activity.

### SUMMARY OF THE INVENTION

An embodiment of the invention relates to a method of isolating restriction endonuclease variants possessing altered specificity and increased specific activity. The method includes obtaining a mutant that has desirable specificity properties. One method is to identify, by structural analysis, critical amino acid residues that make base-specific contact to the recognition sequence or make DNA backbone interactions. This is followed by saturation mutagenesis at the specific residue, i.e. substitution of the wild-type residue to the other 19 amino acid residues. The resulting variants are analyzed in DNA cleavage and binding assays. Another method is to perform random mutagenesis, then screen for mutants that have relaxed specificity by looking for DNA damage in cells that are protected by the cognate methylase. Once variants with altered cleavage or binding specificities are identified, such variants are further mutagenized by random mutagenesis and derivatives with increased cleavage activity are identified by screening in an indicator strain such as the dinD::lacZ indicator strain. High activity pseudorevertants form dark blue colonies on X-gal plates as the result of in vivo DNA damage leading to SOS induction. The process of mutagenizing and screening for increased activity in the dinD::lacZ indicator strain, called recycled mutagenesis, can be repeated to isolate derivatives with even higher activity.

### BRIEF DESCRIPTION OF THE DRAWINGS

FIG. 1A. Analysis of purified wt BsoBI, mutant BsoBI, and pseudorevertant proteins on SDS-PAGE. Lanes 1 to 9, protein size marker, wt BsoBI, D246A, D246S, D246T, D246C, pseudorevertants 9c, 29e, and 8e.

FIG. 1B. Lane 1, protein size marker; Lanes 2 to 7, pseudorevertants 9c protein samples were pre-treated at 4° C., 37° C., 45° C., 52° C., 58° C. and 65° C., respectively. Heat-denatured insoluble proteins were removed by centrifugation.

FIG. 2. Restriction digestion of λ DNA by wt BsoBI, D246A, D246S, D246T, and D246C.

FIG. 2A. Digestion of λ DNA with 2-fold serial dilution of wt BsoBI endonuclease. Lane 1, 3.6 ng BsoBI protein. The specific activity of BsoBI is ˜2×10⁶ units/mg protein.

FIG. 2B. Lanes 1 to 5, partial digestion of λ DNA by BsoBI (0.11 ng), D246A (97 ng), D246S (118 ng), D246T (238 ng), and D246C (775 ng). The large arrow indicates the 1881-bp fragment and the small arrow points to the doublet band. Reaction condition: 1 μg λ DNA incubated with the indicated amount of protein in NEB buffer 2 for 65° C. for 1 h in a total volume of 50 μl (New England Biolabs, Inc., Beverly, Mass.)

FIG. 3. Restriction digestion of specificity testing plasmid pZZ1 by wt BsoBI and variants D246A, D246S, D246T, and D246C.

FIG. 3A. BsoBI digestion of 0.5 μg of AatII-prelinearized pZZ1. The substrate is 2686 bp in length. Digestion at the CCCGGG and CTCGAG sites generated two fragments, 483 bp and 252 bp, respectively. Lane 1, 2-log DNA marker (New England Biolabs, Inc., Beverly, Mass.); lane 2, 25 ng of BsoBI (˜50 units), lanes 3-12, 2-fold serial dilution of 25 ng BsoBI.

FIG. 3B. 3C. 3D. 3E. Variants D246A, D246S, D246T, and D246C digestion of 0.5 μg AatII-prelinearized pZZ1 DNA, respectively. Lane 1, DNA size marker; lanes 2, 0.7 μg of D246A protein; 0.7 μg of D246S, 0.7 μg of D246T, 3 μg of D246C, respectively; Lanes 3-11, 2-fold serial dilution of D246A, D246S, D246T, and D246C proteins, respectively; lane 12, linear DNA substrate.

FIG. 4. Data fitting curve in BsoBI filter binding assay BsoBI binding to the CTCGAG containing oligonucleotides (S1 substrate). The X axis is the final concentration of BsoBI endonuclease. 0.40, 0.80, 1.61, 3.13, 6.25, 12.50, 25.0, 50.0, and 100.0 pM. Oligonucleotide substrate at 2.4 pM. Y axis is F/F₀. Experimental data are shown as filled circles. The line results from data fitting according to equation [1].

FIG. 5. Activity assay using purified pseudorevertant proteins 9c, 29e, and 8e.

FIG. 5A. Digestion of 0.5 mg AatII-prelinearized pZZ1 with variant D246A, wt BsoBI, pseudorevertants 29e and 9c, respectively. D246A, 250 ng protein; wt BsoBI, 29e and 9c, 25 ng protein, respectively.

FIG. 5B. Digestion of 0.5 μg AatII-prelinearized pZZ1 with pseudorevertant 8e endonuclease. Lane1, 2-log DNA size marker; lane 2, wt BsoBI digestion; lane 3, 75 ng of pseudorevertant 8e protein; lanes 4-11, 2-fold serial dilution of 75 ng 8e protein; lane 12, linear DNA substrate. The weak 133-bp fragment is the cleavage product at a “star” site (CCTGGG).

FIG. 6. Restriction digestion of PCR-amplified substrate by PvuII K93R, K93R/R105H, K93R/V50A, and K93R/E18K/R105H. Different amount of proteins (from 10 to 0.03 μg) were used to digest the PCR-amplified DNA substrates at 37° C. for 1.5 hour. Samples were loaded on a 2% agarose gel. Arrows labeled with 1 kb and 0.5 kb are the DNA markers. Arrow “S” indicates the migration of uncut DNA substrate; arrow “MfeI” indicates the migration of the product which has been cleaved at the MfeI site; arrow “PmlI” indicates the migration of DNA product which has been cleaved at the PmlI site.

### DETAILED DESCRIPTION OF THE INVENTION

Restriction endonucleases are here modified so as to have altered specificity. Amino acid substitutions that change the specificity of BsoBI and PvuII endonucleases are first introduced by site-directed mutagenesis or random mutagenesis. The mutants are then subjected to random recycled mutagenesis, and enhanced activity mutants are isolated by screening for DNA damage in a dinD::lacZ indicator strain. Embodiments of the invention involve two steps: step 1 in which low activity mutants with altered specificity are isolated; and step 2 in which mutants in step 1 are further evolved to generate pseudorevertants with higher specific activity.

1. Obtaining Initial Endonuclease Mutant

There are two methods envisioned for obtaining a restriction endonuclease mutation with altered specificity. The first is to engineer one or more amino acid changes by using what is known about the protein:DNA interface. The second is to perform random mutagenesis on the restriction endonuclease gene, and then to devise a selection or screen for mutants with altered specificity.

a. Construction of Variants by Site-Directed Mutagenesis

Amino acid residues that make direct or indirect contacts with target site DNA are identified, for example by protein-DNA co-crystal structure analysis. One or more amino acid residues involved in DNA sequence recognition (making direct hydrogen bond, water-mediated hydrogen bond, or hydrogen bond to the DNA backbone) is replaced by each of the other 19 amino acids via site-directed mutagenesis (Morrison and Desrosiers, BioTechniques 14:454-457 (1993)). Variants are isolated and their specificity is assayed by in vivo and/or in vitro methods as described below. In the embodiment of the invention practiced here (Example I), the co-crystal structure of BsoBI-DNA indicated that residue Asp246 of BsoBI endonuclease makes a water-mediated hydrogen bond to the degenerate purine (adenine) in the BsoBI recognition sequence. By replacing the Asp codon at 246 with codons for the other 19 amino acids via site-directed mutagenesis (inverse PCR mutagenesis, Ochman et al. Bio/technology, 759-760 (1990)), BsoBI variants with altered binding or cleavage specificity were isolated. Most of the variants are null mutants without binding and cleavage activity. However, D246A, D246S, D246T, and D246C variants display low, but detectable cleavage activity at 65° C. on λ and pUC19 substrates. Moreover, purified D246A, D246S, and D246T proteins show much reduced cleavage activity at the CTCGAG site when CCCGGG and CTCGAG sites are present on the same DNA substrate. The variants also displayed reduced cleavage activity at the CTCGAG site on λ and φX174 DNA substrates. The partial digestion pattern of D246C is also different from that of the wt enzyme, with predominant cleavage at the CCCGGG site under limited digestion conditions. BsoBI variants D246A, D246S, and D246T changed specificity toward XmaI (C/CCGGG). The specific activity of D246A is approximately 0.7% of the wt enzyme on the CCCGGG substrate.

b. Isolation of Mutants by Random Mutagenesis and Screening

A random or semi-random mutant library of the restriction endonuclease gene is created, for example by PCR mutagenesis, cassette mutagenesis, chemical mutagen treatment such as sodium bisulfite, nitrosoguanidine, and hydroxylamine, or by passing through a hyper-mutator strain such as mutD deficient strain, or by UV mutagenesis. The mutant library is ligated into an expression vector, and the vector library is used to transform a bacterial indicator strain. The indicator strain contains the dinD::lacZ allele along with a methylase that protects against the wild-type endonuclease. In this case, cells expressing variants that cleave sites other than the wild-type site sustain DNA damage, and appear blue on X-gal plates. In the embodiment of the invention practiced here (Example II), PCR mutagenesis of the gene encoding PvuII followed by screening with this method yielded a mutant with Lys residue 93 changed to Arg. This K93R mutant had relaxed specificity, as evidenced by cleavage assays on λ and pUC19 DNA, but had a catalytic efficiency, when measured at a PvuII site, that was reduced approximately 50-fold from wild-type PvuII.

C. Characterization of Initial Mutant.

The specificity of the initial mutant can be characterized by a number of methods. An example of an in vitro method is to assay the cleavage activity of variants on DNA substrates of known sequence and assess the pattern of cleavage. The cleavage activity of the variants is assayed using cell extracts or purified protein from the variant cells. If necessary to assay the activity, the mutant proteins are purified to near homogeneity, for example by affinity, ion exchange, and/or gel filtration chromatography. Another in vitro method useful to characterize the DNA binding properties of variant endonucleases is by DNA mobility shift assays or filter binding assays, which allow measurement of the dissociation constant of the variant from an oligonucleotide of defined sequence. An example of an in vivo method is to express the mutant restriction endonuclease in a cell containing the dinD::lacZ allele along with a methylase that modifies a sequence or set of sequences related to the wild-type site of the restriction endonuclease. In this case, cells expressing variants that cleave sites other than the wild-type site appear blue on X-gal plates.

2. Isolation of Pseudorevertants with Enhanced Activity by Recycled Mutagenesis and in vivo SOS Induction

The variants identified in (1.) above usually have reduced cleavage activity because of the amino acid substitution(s). A laboratory evolution strategy is employed to isolate pseudorevertants that have enhanced catalytic activity. Such recycled mutagenesis can be repeated until a desired specific activity is achieved.

Plasmids carrying coding sequences of the restriction endonuclease variants (variant alleles) can be mutagenized by any of a number of methods, for example by error-prone PCR, cassette mutagenesis, chemical mutagen treatment such as sodium bisulfite, nitrosoguanidine, and hydroxylamine, by UV mutagenesis, or by passing through a hyper-mutator strain such as mutD deficient strain. The plasmid library carrying the mutant genes is used to transform an indicator stain such as the dinD::lacZ strain, which, if desired, can contain one or more DNA methylases to constrain the set of sequences where DNA damage is assessed. The plasmid carrying the mutant endonuclease library and the conditions on the plate are designed so that the original variant yields a white or light blue colony on X-gal plates with appropriate antibiotics and inducer concentration. For example, the plasmid could have a lac promoter that expresses the mutant endonucleases, and the X-gal plate could have a sub-optimal concentration of IPTG. Mutants that have higher catalytic activity create dark blue colonies. To maintain the genetic stability of such pseudorevertants and to prevent further mutation, blue colonies are boiled and the released DNA is used to transform protected cells or partially protected cells by the cognate or non-cognate methylases.

This restriction endonuclease engineering method is not limited to one round of random mutagenesis and screening. Those skilled in the art may go through more than one round of mutagenesis and screen for active endonuclease that have similar or greater catalytic activity than the wild-type enzyme.

Although the above-outlined steps represent the preferred method for practicing the present invention, it will be apparent to those skilled in the art that the above-described approach can vary in accordance with techniques known in the art.

The following two Examples are given to illustrate embodiments of the present invention as it is presently preferred to practice. It will be understood that the two Examples are illustrative, and that the invention is not to be considered as restricted thereto except as indicated in the appended claims.

The references cited above and below are herein incorporated by reference.

### EXAMPLE I

### Recycled Mutagenesis of BsoBI to Generate BsoBI Variants with Increased Specificity and Enhanced Catalytic Activity

1. Site-Directed Mutagenesis of BsoBI Residue D246, Variant Protein Purification, and Cleavage Activity Assay

Strains and plasmids: The dinD::lacZ indicator strain ER1992 was described previously (U.S. Pat. No. 5,498,535). ER2683 was constructed and provided by E. Raleigh (New England Biolabs, Inc., Beverly, Mass.). Plasmid pACYC-bsoBIM and pRRS-bsoBIR have been described (Ruan, et al. Gene 188:35-39 (1977)). All BsoBI variants were expressed from P_{lac} on pRRS, a pUC derivative. The double mutant M130A/D246A was constructed in the intein vector pTYB1 (New England Biolabs, Inc., Beverly, Mass.). Oligonucleotides were synthesized by New England Biolabs, Inc. Organic Synthesis Division (Beverly, Mass.). Plasmid DNA was isolated by Qiagen spin column (Valencia, Calif.). All desired mutations were confirmed by DNA sequencing using the AmpliTaq dideoxy terminator sequencing kit and an ABI 373A sequencer. The complete alleles were sequenced to detect unwanted mutations.

Transformation: 10 to 100 ng of plasmid DNA was incubated with 100 to 200 μl competent cells on ice for 30 min. The cells were subjected heat shock at 37° C. for 3-5 min. Following incubation at room temperature for 5 min, equal volume of LB or SOB were added to the cells and incubated at 37° C. for 1 h to amplify the transformed cells. Transformants were incubated at 30° C. to 37° C. overnight on X-gal plates plus Ap (100 μg/ml). Incubation of transformants of BsoBI variants at low temperature tends to stabilize the mutant-carrying plasmids presumably the enzyme displays lower activity at the low temperature in vivo.

Electroporation: 10 ng to 50 ng DNA was mixed with 50 μl to 100 μl electro-competent cells and the cells were immediately transferred to pre-chilled cuvets. The cells were electro-shocked in a Gene Pulser (Bio-Rad, Richmond, Calif.) with 25 μF capacitor, 2.5 kV, and the pulse controller unit to 200 Ω. The cells were diluted in 0.5 ml to 1 ml of TB or LB broth and incubated at 37° C. for 1 h. Following amplification 100 μl to 200 μl cells were plated on X-gal plates with Ap and incubated at 37° C. overnight. Chemically competent cells were prepared CaCl₂treatment. Electro-competent cells were prepared by the standard procedures except that 10% glycerol was included in all washing steps of cold sterile distilled water.

Protein purification: BsoBI and its variants were produced in ER2683 [pACYC-BsoBIM, pRRS-BsoBIR (R*)]. The procedure for IPTG-induction and cell lysis was followed as previously described (Xu and Schildkraut J. Biol. Chem. 266:4425-4429 (1991)). The cell lysate was heated at 65° C. for 1 h and heat-denatured E. coli proteins were moved by centrifugation (the heat-treatment step was omitted for pseudorevertant 9c purification). The BsoBI endonuclease or each variant was further purified by chromatography through Heparin-Sepharose columns (Pharmacia, Piscataway, N.J.). Proteins were eluted in a NaCl gradient of 50 mM to 1 M in 10 mM Tris-HCl, pH 7.5. Other protein/DNA/RNA was further removed by passing through a DEAE-Sepharose column. Final products of BsoBI or each variant were analyzed on SDS-PAGE and purity was estimated at >90% homogeneity. The purity of 9c pseudorevertant was estimated at 50%. The double mutant M130A/D246A was purified from a chitin column and the fusion protein was cleaved on the column by addition of DTT overnight at 4° C. BsoBI and each variant were stored in 50% glycerol at −20° C.

Determination of substrate specificity for BsoBI variants: BsoBI digestion was performed in NEB buffer 2 at 65° C. for 1 h. The products were analyzed on 0.7% to 1% agarose gels (New England Biolabs, Inc., Beverly, Mass.). The substrates for BsoBI and each variant were λ DNA (8 BsoBI sites), plasmid pZZ1 with two BsoBI sites (CCCGGG and CTCGAG), pUC19 DNA (CCCGGG), and φX174 DNA (CTCGAG). Another subset of BsoBI substrates was derived from pUC19 in which the BsoBI site CCCGGG was mutated to CTCGAG, CCCGTG (CTCGGG) by inverse PCR mutagenesis with two mutagenic primers, DpnI digestion and transformation. The same flanking sequence of the BsoBI site was thus maintained (gta-BsoBI site-gat). AatII-prelinearized plasmid was digested with BsoBI or variants D246A, D246S, D246T, D246C and the digestion products were analyzed by agarose gel electrophoresis.

There are a total of 18 hydrogen bonds to the bases of CTCGAG sequence and one of the hydrogen bonds involves a water-mediated contact between the D246 side chain and the adenine (ctcgAg). D246 was substituted to all 19 other amino acids via site-directed inverse PCR mutagenesis. Mutant alleles were confirmed by DNA sequencing. Cell extracts were prepared for all 19 variants and cleavage activity was tested on λ DNA at 65° C. and 37° C. Wt BsoBI displays 5 times more cleavage activity at 65° C. than at 37° C. When cell extracts of all 19 variants were assayed on λ DNA, it was found that cell extracts containing D246C, D246S, D246T, and D246A displayed partial cleavage activity at 65° C. All other variants showed no detectable cleavage activity on λ or pUC19 DNA (<0.001% activity, data not shown).

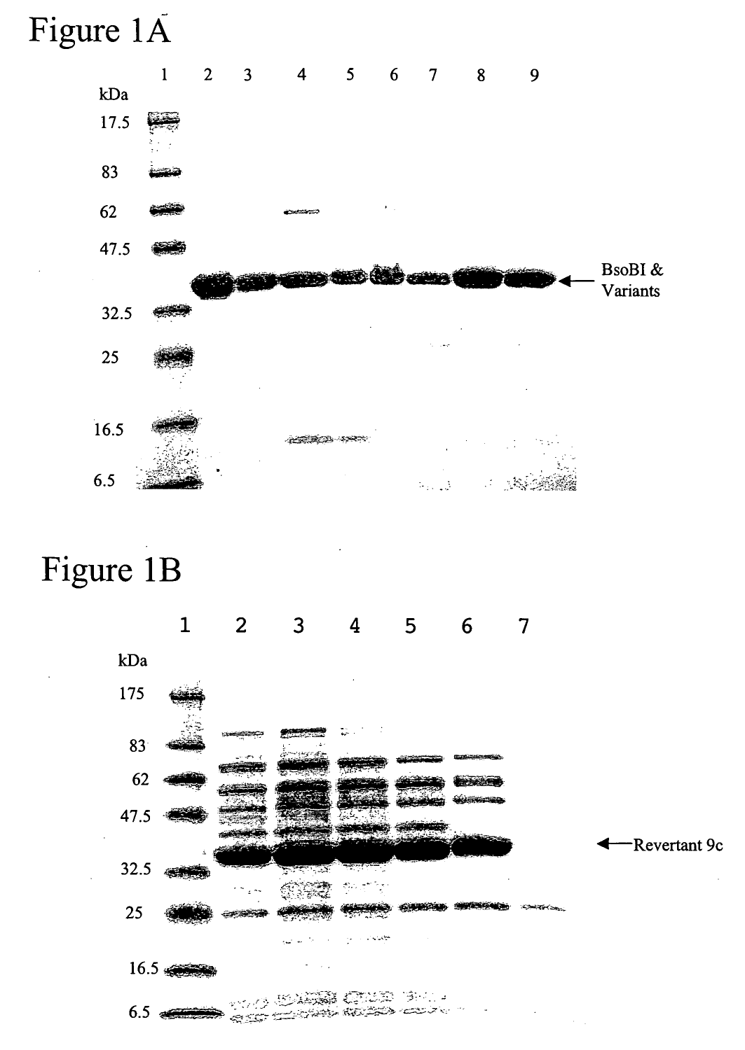

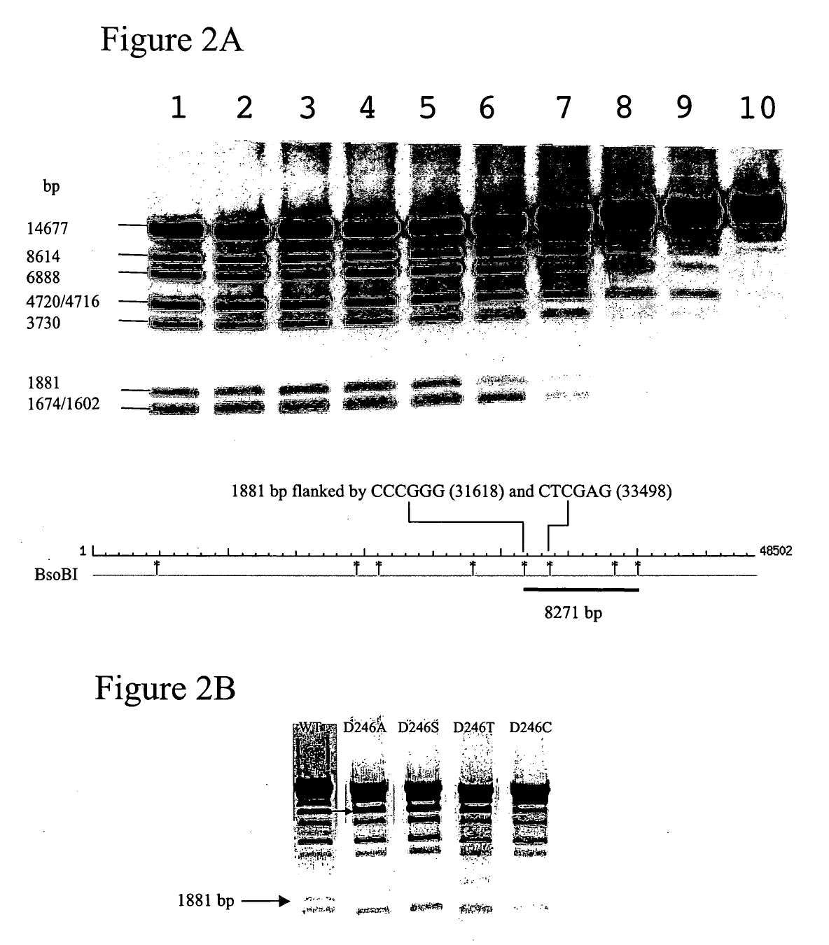

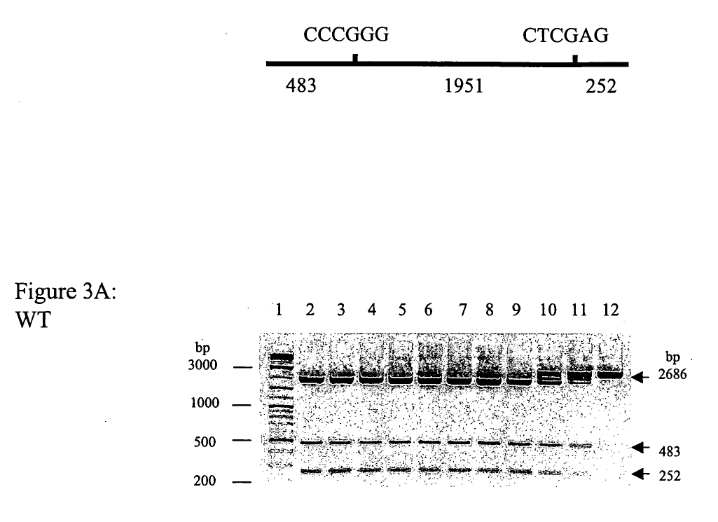

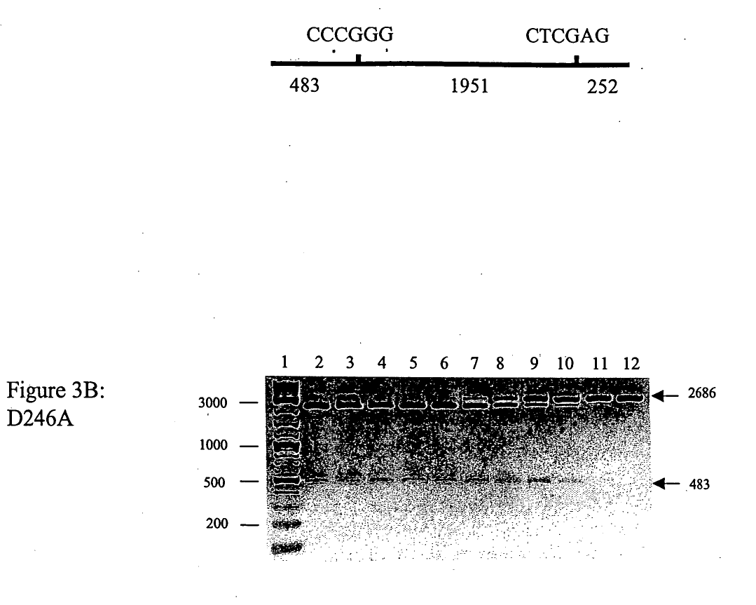

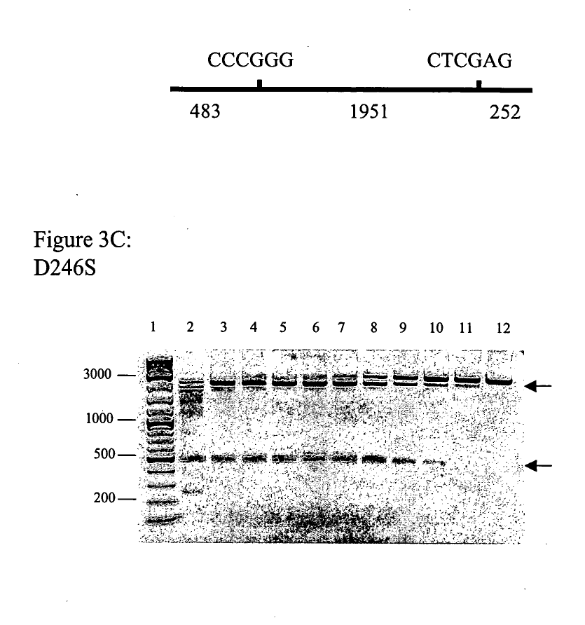

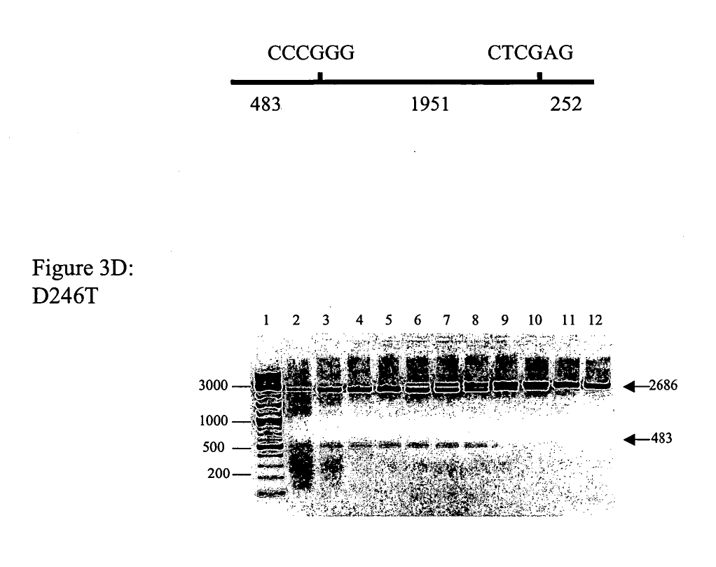

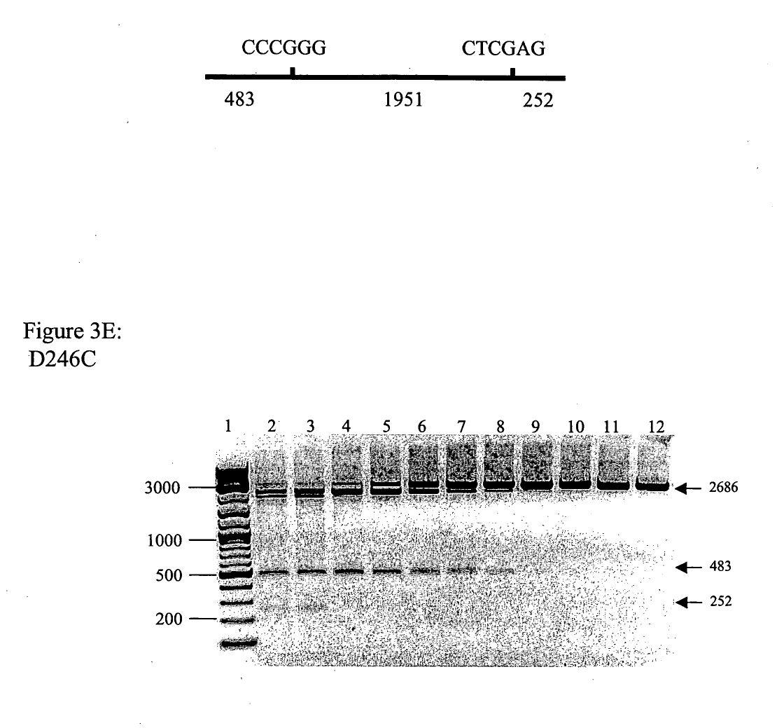

The four partially active variant proteins were purified to more than 90% purity through heat-treatment and chromatography and analyzed on SDS-PAGE (FIG. 1A, lanes 3-6). The cleavage activities of BsoBI, D246A, D246S, D246T, and D246C were detected on λ DNA (FIG. 2A, 2B). The partial digestion pattern of the four variants appeared different from that of the wt enzyme (FIG. 2B). Inspection of the subset of BsoBI sites on λ DNA indicated that an 1881-bp fragment (flanked by CTCGAG and CCCGGG sequences) was missing from the DNA products, suggesting that the D246A variant failed to cleave one of the sites. Since the other fragments flanked by CCCGGG sites were cleaved and there is only one CTCGAG site in λ DNA, it was suspected that the four variants did not cleave the CTCGAG site efficiently. The failure to cleave CTCGAG created a doublet band (8271 bp/8614 bp, FIG. 2B, indicated by a small arrow) when λ DNA was digested by D246A, D246S, and D246T (the doublet was less prominent in D246C). To directly compare the cleavage efficiency of CCCGGG and CTCGAG sites, a pUC19 derivative, pZZ1, was constructed that contains both symmetric sites and was used in cleavage assay (flanking sequences, gta-CCCGGG-gat, gtg-CTCGAG-ggg). FIG. 3 shows the digestion results of wt BsoBI, D246A, D246S, D246T, and D246C on pre-linearized pZZ1 substrate. BsoBI endonuclease cleaves CCCGGG and CTCGAG with almost equal efficiency, generating two cleaved products 252 bp and 483 bp (FIG. 3A, lanes 2 to 11). However, D246A generated only a 483 bp product at low enzyme concentration (FIG. 3B, lanes 3 to 11), indicating preferential cleavage at the CCCGGG site. On the two-site substrate (pZZ1), the cleavage rate of D246A on the CCCGGG site is at least 64 times faster than the CTCGAG site. The cleavage activity of D246S is comparable to D246A while D246T displays approximately ˜4-fold lower activity than D246A with the same preferential cleavage of the CCCGGG site (FIG. 3, C and D). The cleavage pattern generated by D246C was also different from the wt enzyme. Cleavage of the CCCGGG site was detected first at low enzyme concentration and cleavage at the CTCGAG site was observed at higher enzyme concentration (FIG. 3E). D246C also generated some “star” fragments (FIG. 3E, lanes 2 and 3).

To further confirm the altered specificity of D246A substitution, a double mutant M130A/D246A was constructed as a fusion protein containing an intein with a chitin binding domain. BsoBI variant M130A eliminates the internal initiation codon ATG at position 130 and thus the internally initiated 22-kDa BsoBI_{S} protein is no longer translated. M130A maintains the wt enzyme specificity although its specific activity is reduced 4-fold (˜5×10⁵ u/mg). The double mutant protein M130A/D246A was purified to homogeneity through a chitin column and intein cleavage was initiated by addition of DTT. M130A/D246A also displays much reduced cleavage activity at the CTCGAG site on pZZ1 substrate; only cleavage at the CCCGGG site was detected.

2. Filter Binding Assay

The following oligonucleotides were synthesized and gel-purified as substrates for BsoBI filter binding assay:

| 5′ GAGTGTTATGGGTAGCTCGAGTGAAGTGGGAATATC | (#1, symmetric BsoBI site) | (SEQ ID NO:1) |
|---|---|---|
| 5′ GATATTCCCACTTCACTCGAGCTACCCATAACACTC | (#2) | (SEQ ID NO:2) |
| 5′ GAGTGTTATGGGTAGCCCGGGTGAAGTGGGAATATC | (#3, symmetric BsoBI site) | (SEQ ID NO:3) |
| 5′ GATATTCCCACTTCACCCGGGCTACCCATAACACTC | (#4) | (SEQ ID NO:4) |
| 5′ GAGTGTTATGGGTAGCTCGGGTGAAGTGGGAATATC | (#5, non-symmetric BsoBI site) | (SEQ ID NO:5) |
| 5′ GATATTCCCACTTCACCCGAGCTACCCATAACACTC | (#6) | (SEQ ID NO:6) |
| 5′ GAGTGTTATGGGTAGCATAACTGAAGTGGGAATATC | (#7, non-specific site) | (SEQ ID NO:7) |
| 5′ GATATTCCCACTTCAGTTATGCTACCCATAACACTC | (#8) | (SEQ ID NO:8) |

Duplex DNA substrates were prepared by pairing oligonucleotides #1 and #2, #3 and #4, #5 and #6, #7 and #8 in equal molar ratio. The DNA was first heated at 95° C. for 30 min and then kept at 55° C. for 30 min and gradually cooled down to room temperature. The duplex DNA substrates were termed S1=#1+#2 (CTCGAG), S2=#3+#4 (CCCGGG), S3=#5+#6 (CCCGAG/CTCGGG), and N=#7+#8 (CATAAC/GTTATG). The dsDNA substrates were further purified by passage through Qiagen spin columns.

The four DNA substrates were end-labeled by γ-³³P ATP (PE Life Sciences, Boston, Mass.) with T4 polynucleotide kinase and then purified by passage through Qiagen spin columns. The binding condition was in 10 mM Tris-HCl, pH 7.5, 100 mM NaCl, 50 mg/ml BSA, 4 mM CaCl₂ in 200 ml volume. The labeled DNA substrates were diluted to a final scintillation count of 1000-5000 cpm and varying amount of BsoBI or each variant was added. Following incubation at room temperature for 10 min, the bound complex was loaded onto Micropure EZ column (Millipore Corporation, Bedford, Mass.) and spun at 16K g for 1 min. Protein and protein:DNA complexes are bound to the membrane in the Micropure EZ column and only the free unbound DNA is in the flow-through fraction. 180 μl of the flow-through liquid was immediately transferred to scintillation counting vials filled with 1.5 ml OPTI-FLUOR for aqueous samples (Packard, Meriden, Conn.). The radioactivity count from the sample without BsoBI is F₀, and the radioactivity count from sample with BsoBI or each variant is F. The apparent K_{d} value is calculated from F/F₀ and the BsoBI concentration [E] according to equation [1].
F/F₀=a [K_{d}/([E]+K_{d})]+b [1]
a and b are equation fitting parameters, where b is the non-specific flow-through, and a is the maximum change amplitude.

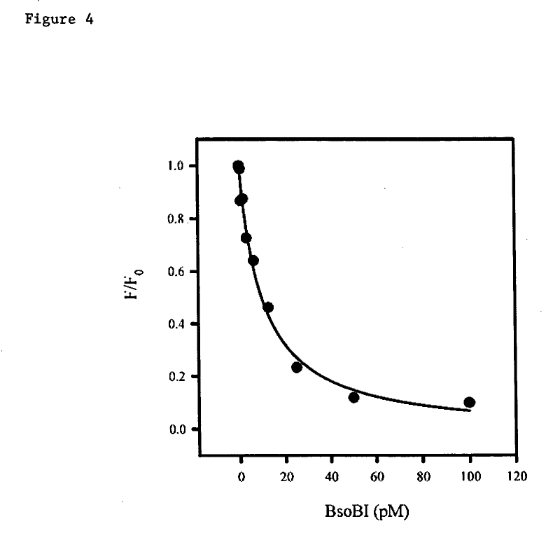

DNA binding properties of BsoBI endonuclease and variants: To obtain reproducible BsoBI:DNA binding data, a filter binding assay utilizing a microcentrifuge filter was developed. In the control experiment, more than 99% of the wt BsoBI endonuclease was retained on the filter of the spin column. When labeled duplex DNA was loaded onto the membrane in the absence of protein, more than 95% of the DNA passed through the column. Therefore, one can measure the amount of unbound DNA (free DNA) that passes through the column, calculate the bound DNA and fit the data into equation [1] to determine the K_{d} value (FIG. 4) BsoBI endonuclease, D246A, D246C, D246E, D246R, and D246Y proteins were purified to >90% homogeneity by heat-treatment of the cell extract followed by chromatography. The heat-treatment step removed approximately 80% of E. coli proteins. The filter-binding assay was performed for wt BsoBI endonuclease and variants D246A, D246C, D246E, D246R, and D246Y in the presence of Ca⁺⁺. The inclusion of Ca⁺⁺ in the binding buffer stabilizes the protein:DNA complex in DNA mobility shift assays (data not shown) and it was presumed that Ca⁺⁺ does the same in the filter binding assay. The K_{d} values of wt BsoBI for all three subset sites CTCGAG, CCCGGG, and CCCGAG are 9.2 pM, 6.6 pM, and 5.3 pM, respectively, indicating that BsoBI binds to CCCGGG and CCCGAG with slightly higher affinity than the CTCGAG site. The K_{d} value for non-specific duplex DNA is 1.4 nM, a 219-fold reduction in binding affinity compared to the CCCGGG site (Table 1 and 2).

TABLE 1
K_{d} value of BsoBI and its variants on BsoBI sites
(CTCGAG, CCCGGG, and CCCGAG) and a non-BsoBI
site (CATAAC)

| BsoBI[n]^{a} | CTCGAG | CCCGGG | CCCGAG | CATAAC |
|---|---|---|---|---|
| WT[6] | 9.2^{b} (±2.0)^{c} | 6.6 (±1.2) | 5.3 (±0.9) | 1445 (±143) |
| D246A[2] | 219 (±40) | 3.1 (±0.6) | 17 (±4) | 578 (±97) |
| D246C[3] | 74 (±11) | 30 (±4) | 64 (±9.8) | 142 (±10) |
| D246E[3] | 80000 (±20000) | 2840 (±683) | 42050 (±1578) | 93200 (±3347) |
| D246R[3] | 89 (±20) | 38 (±3.5) | 39 (±4.6) | 64 (±4.9) |
| D246Y[3] | 132 (±16) | 41 (±2) | 57 (±4) | 449 (±67) |

^{a}Number in bracket indicates the number of experiments.
^{b}Average K_{d} value in [pM].
^{c}Number in parenthesis indicates standard error from data fitting. Binding substrate, [³³P] labeled 36-bp duplex oligonucleotides with the same flanking sequences.

TABLE 2
Ratio of relative K_{d} values (K_{d} of individual BsoBI
site or non-specific site normalized relative to K_{d} of
CCCGGG)

| BsoBI | CTCGAG | CCCGGG | CCCGAG | CATAAC |
|---|---|---|---|---|
| WT | 1.4 | 1 | 0.8 | 218.9 |
| D246A | 70.6 | 1 | 5.5 | 186.5 |
| D246C | 2.5 | 1 | 2.1 | 4.7 |
| D246E | 28.2 | 1 | 14.8 | 32.8 |
| D246R | 2.3 | 1 | 1.0 | 1.7 |
| D246Y | 3.2 | 1 | 1.4 | 11.0 |

When D246 is replaced by a Glu residue with a longer side chain, a possible outcome may be expulsion of the water molecule and direct recognition of either A or G in the degenerate position. However, the purified D246E protein lacked measurable cleavage activity and also displayed low DNA binding affinity. The relative affinity was about 30 times higher for CCCGGG as compared to the CTCGAG and CATAAC sites. The low affinity for CTCGAG is about the same as the non-BsoBI site CATAAC (K_{d}=80 nM and 93.2 nM, respectively, Table 1 and 2). It was concluded that the variant D246E has lost selective binding to CTCGAG, but partially retained the selectivity toward the CCCGGG site. The other two variants with conservative amino acid changes, D246N and D246Q, displayed no detectable cleavage activity in cell extracts (<0.001% of wt activity). The variants D246R and D246Y show reduced binding affinity to BsoBI sites and increased binding affinity to non-specific DNA.

Variant D246A shows moderately reduced affinity to CCCGAG (CTCGGG) while binding to the CTCGAG site is severely reduced, which is consistent with the DNA cleavage data. Thus, the reduced cleavage activity of D246A on the CTCGAG substrate was most likely the result of impaired binding to this site.

3. Isolation of D246A Pseudorevertant by Random Mutagenesis (Error-Prone PCR)

One set of inverse PCR primers was synthesized to amplify and mutagenize the D246A allele. The PCR primers were designed to anneal to the β-lactamase gene (Ap^{R}) and have the following sequences:

(SEQ ID NO:9)

5′ GCTGAAGATCAGTTGGGTGCTCGAGTGGGTTACATCGAACTG 3′

(SEQ ID NO:10)

5′ CAGTTCGATGTAACCCACTCGAGCACCCAACTGATCTTCAGC 3′

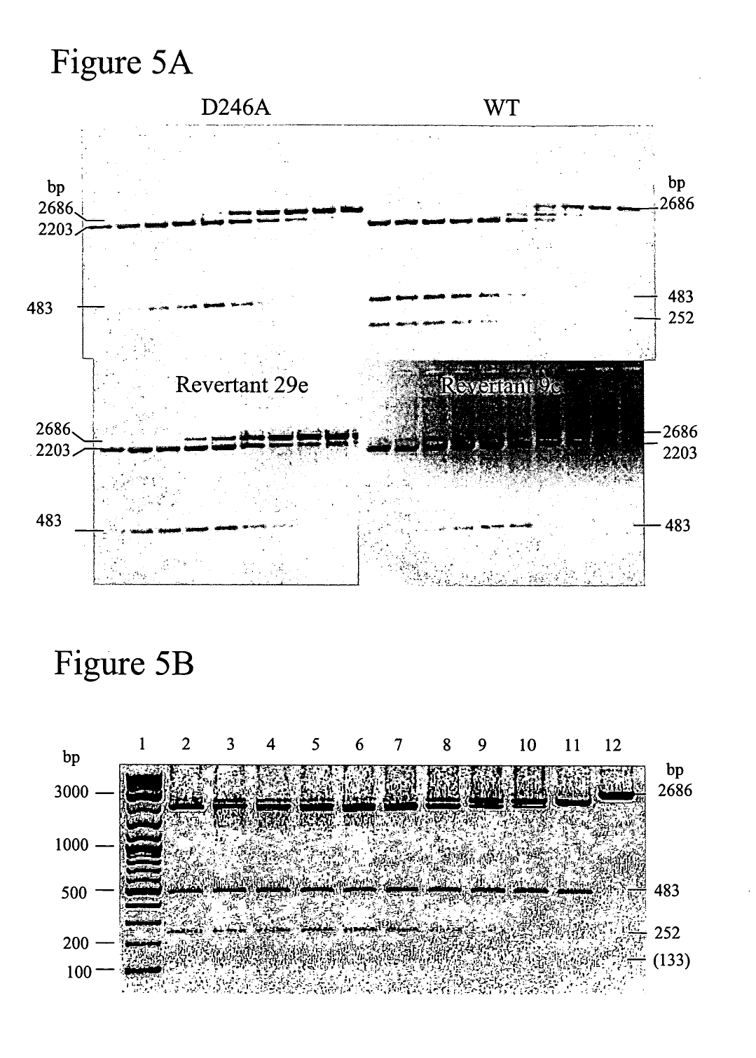

Error-prone PCR mutagenesis conditions were 95° C. 5 min for 1 cycle; 95° C. 30 sec, 55° C. 30 sec, 72° C. 4 min, for 30 cycles with Vent® (exo⁻) DNA polymerase under non-equal dNTP concentration (1 mM dCTP and dTTP, 0.2 mM dATG and dGTP). Following DpnI digestion of the template DNA (DpnI digests G^{m}ATC methylated template DNA), the PCR products were transferred into a dinD::lacZ indicator strain (ER1992) by transformation or electroporation and Ap^{R} transformants were plated on LB agar plus Ap and X-gal at 37° C. overnight. Plasmid DNA was isolated from blue colonies and subjected to restriction mapping. The D246A allele was completely sequenced to determine additional mutation(s). Cell extracts from dark blue colonies were also prepared and used to digest λ or pZZ1 DNA to determine substrate specificity. pseudorevertant protein expression was induced with IPTG at 37° C. and proteins were partially purified at 4° C. Cleavage activity was assayed at 65° C. For temperature-sensitive pseudorevertant, activity was assayed at 37° C. and 65° C. DNA double strand breaks induce the SOS response in E. coli and thereby increase β-galactosidase expression from dinD::lacZ gene fusions. The dinD::lacZ indicator strain ER1992 carrying the D246A allele on pRRS forms light blue colonies on X-gal plates. The D246A allele was further mutagenized by error-prone inverse PCR and those amino acid substitutions that increase the specific activity of the enzyme were identified. The dark blue colonies were screened for more active pseudorevertants. To maintain the genetic stability of such pseudorevertants and to prevent further mutation, blue colonies were boiled and the released DNA was used to transform M.BsoBI protected cells. The inclusion of M.BsoBI was critical because some dark blue pseudorevertants were difficult to amplify in liquid cultures. In some cases, non-cognate methylases conferring partial protection can also be used to increase the stability of the pseudorevertant clones. Cell extracts were prepared from clones derived from dark blue colonies and cleavage specificity was determined on λ and pZZ1 DNAs. Three types of pseudorevertants were found among the blue colonies. The coding sequence of the six most active pseudorevertants not containing IS elements was sequenced and the predicted amino acid substitutions are shown in Table 3. The predominant class I (>95%) were insertion element containing mutants that contained IS5 upstream of the D246A allele. The three pseudorevertants in class II maintained the site preference of D246A (FIG. 5A, Table 3, isolates 9c, 11c, and 29e). Isolate 29e carries a secondary site amino acid substitution (D246A/C317W). Isolates 9c and 11c are the same and carry six additional amino acid substitutions (R38S/L62I/D112Y/N169T/L238F/D246A/A248S). Despite the significant suppression effect of the new mutations in the class II pseudorevertants, most of the new amino acid changes are located outside of the DNA:protein interface. Only residue A248 is involved directly in DNA recognition by hydrogen bonding to the external cytosine in the recognition sequence. When the mutation in 29e (C317W) was combined with 9c to generate variant 9c/29e (R38S/L62I/D112Y/N169T/L238F/D246A/A248S/C317W), the resulting enzyme did not display enhanced cleavage activity as compared to 29e. We therefore conclude that C317W substitution is an independent pathway to increase D246A activity since there is no apparent additive effect between 9c and 29e.

The exact contribution of each suppressor in 9c is not clear since 9c carries 6 additional amino acid substitutions. However, the non-contributing residues have been initially identified. When S38, I62, T169, and F238 were returned to the wt residue by site-directed mutagenesis, the resulting variants 9c/R38, 9c/L62,

TABLE 3
Activity and Specificity of D246A revertants

| | | | Relative |
|---|---|---|---|
| | | Cleavage | activity |
| Enzyme | Amino Acid Substitutions | of CTCGAG^{a} | (65° C.)^{b} |
| Wt BsoBI | — | yes | 100% |
| D246A | D246A | No | 0.5%-0.8%^{c} |
| 9c, 11c | R38S/L62I/D112Y/N169T/ | No | 10% |
| | L238F/D246A/A248S | | |
| 29e | D246A/C317W | No | 4% |
| 6c | E46V/D61E/S94T/D246A | Yes | 4% |
| 8d | N36K/D246A | Yes | 5% |
| 8e | A32T/T40P/Q140L/D246A | Yes | 11% |

^{a}Cleavage of CTCGAG site was assayed on pZZ1.
^{b}Relative cleavage activity was measured on CCCGGG site on pUC19.
^{c}D246A protein preparations 1 and 2 display 0.8% and 0.5% of wt activity, respectively (assayed on pUC19 substrate).

9c/N169, and 9c/L238 displayed cleavage activity similar to 9c, indicating that substitutions S38, I62, T169, and F238 play a limited role in increasing D246A activity. Therefore, the enhancement of D246A activity could be attributed to substitutions of D112 by Y and A248 by S. However, determination of the individual contribution of Y112 and S248 was problematic. Elimination of S248 (reversion back to the wt residue Ala) greatly reduced the mutant enzyme's activity, but the double mutant D246A/A248S was inactive (data not shown). Thus, the D112Y and A248S substitutions may act synergistically to “revive” the D246A allele.

The three pseudorevertants in class III regained the ability to cleave CTCGAG site on λ NA (Table 3). Isolate 8d carries one additional amino acid substitution N36K (N36K/D246A). Isolate 8e carries multiple amino acid changes at residues 32, 40, 140 (A32T/T40P/Q140L/D246A). Isolates 6c, 8d and 8e display 4%, 5%, and 11% of wt specific activity, a 6- to 16-fold increase compared to 0.7% activity of D246A (FIG. 5B, Table 3). Purified 8e protein cleaved both CCCGGG and CTCGAG sites (FIG. 5B, lanes 3-10). At higher enzyme concentration, it also generated a small fragment (˜133 bp), indicative of cleavage at a BsoBI “star” site, CCTGGG (FIG. 5B, lanes 5 and 6).

The pseudorevertant blue phenotype revealed in vivo activity at 37° C. However, the cleavage activity was assayed in vitro at 65° C. BsoBI is thermophilic and more active at 65° C. than 37° C. (100% activity at 65° C. and 20% activity at 37° C., data not shown). It was noted that 9c displays cleavage activity at both 37° C. and 65° C. However, pretreatment of 9c at 65° C. for 1 h resulted in complete loss of cleavage activity in subsequent activity assay. Presumably the cleavage activity detected at 65° C. occurs rapidly before the protein is fully heat-denatured. The partially purified 9c protein is resistant to heat-denaturation at 45° C. to 58° C. (FIG. 1B, lanes 3-6), but the protein was fully inactivated (denatured) after 1 h heat-treatment at 65° C. (FIG. 1B, lane 7). The other five pseudorevertants did not display temperature-sensitive phenotype.

### EXAMPLE II

### Isolation PvuII Mutants with Increased Specificity and Increased Catalytic Activity

Media: LB media contains 10 g/L Tryptone (Difco), 5 g/L yeast extract (Difco), 5 g/L NaCl, 1 g/L glucose, and 10 mM MgSO₄. LB agar plates contained 15 g Difco agar/L. Ampicillin (amp) was used at 100 μg/ml; chloramphenicol (cam) was used at 34 μg/ml; kanamycin (Kan) was used at 50 μg/ml. Isopropylthiogalactoside (IPTG) was used at concentrations of 0.01 to 1 mM, as noted in the text.

Strains and plasmids: The dinD::lacZ strain ER1992 has been described previously. Strain ER2745 is a derivative of ER1992 that contains the T7 RNA polymerase under the lac UV5 promoter, integrated into the chromosome (International Publication No. WO 99/64632). PR1553 is a derivative of ER2745 carrying the PvuII methylase on plasmid pPR1551. Plasmid pPR1551 is a derivative of pBBA5 (Balendiran, et al. Proteins: Structure, Function and Genetics 19:77-79 (1994)) which arose spontaneously as a chloramphenicol-resistant kanamycin-sensitive variant. Plasmid pLT7K was described previously (Kong, et al. Nucl Acids Res 28:3216-3223 (2000)) and was further modified by inserting a linker, 5′-GGATCCGAGCTCGAATTCGCGGCCGCACACCACCACCACCACC ACTAGACTAGTGGCCAGGCCGGCC-3′(SEQ ID NO: 11) between the BamHI and XhoI sites. The resulting plasmid is named pLT7KH, and is designed to facilitate subcloning of R.PvuII-His-tag fusion genes. The same linker was also inserted between the BamHI and SspI site of pUC19 to yield pCH1. Plasmids pLT7KH-pvuIIR and pCH1-pvuIIR were constructed as follows: Two primers were designed to use amplify the pvuIIR gene, 5′-AGTCGGATCCGAGCTCTTAAAGGAACACGAAAA TGAGTCACCCAGATC-3′ (SEQ ID NO: 12) and 5′-AGTCGAATTC GTAAATCTTTGTCCCATGTTCCATTAC-3′ (SEQ ID NO: 13). These primers were used to amplify the pvuIIR gene from pBBE3 (Balendiran, et al. Proteins: Structure, Function and Genetics 19:77-79 (1994)). The resulting fragment was cleaved with BamHI and EcoRI and inserted into pLT7KH and pCH1 cleaved with the same two enzymes.

Plasmid preparation: Plasmid DNA was prepared using the Qiaprep Spin Miniprep Kit (Qiagen, Studio City, Calif.), according to the manufacturer's instructions.

DNA sequencing: DNA sequencing reactions were performed on Applied Biosytems (Foster City, Calif.) automated DNA sequencers (Models 373, 377 and/or 3100) using BigDye™ labeled dye-terminator chemistry. Oligonucleotide primers were synthesized at New England Biolabs, Inc. Organic Synthesis Division (Beverly, Mass.) using Applied Biosystems synthesizers (Models 392, 394 or Expedite 8909 with MOSS). Crude oligonucleotides were diluted to 3.2 pmol/μl in double distilled sterile MilliQ™ (Millipore, Bedford Mass.) water and 1 ul was used in the sequencing reaction. 500 ng of plasmid template DNA were utilized in the sequencing reactions. Reaction volumes were 10 μl, containing 4 μl of Applied Biosystems BigDye™ premix. Reactions were built with a Biomek® 2000 Robot (Beckman Instruments, Palo Alto, Calif.) in 96-well plates. The reaction components were transferred directly into a 96 well tray on an MJ thermal cycler (MJ Research, Watertown, Mass.) (with a Power Bonnet™ heated lid) attached to the robot, and thermal cycled immediately upon addition of all reagents. Cycling conditions were: 1 step of 96° C. for 4 min followed by 25 cycles of 96° C. for 30 sec, 50° C. for 15 sec and 60° C. for 4 min. For BACs and large phage templates, cycling conditions were 1 step of 96° C. for 4 min followed by 35 cycles of 96° C. for 10 sec, 50° C. for 5 sec and 60° C. for 4 min.

Following thermal cycling, excess dye-terminators were removed with Centrisep® spin columns (Princeton Separations, Adelphia, N.J.) on an individual reaction basis, as described in the product manual, or in a 96 well system (Edge Biosystems, Gaithersburg, Md.). For the EDGE plates, cycled sequencing reactions were directly pipetted onto the center of each well of a 96 well separation plate, with a gel bed of hydrated Sephadex mixture. The plate was centrifuged at 800 rpm in a Beckman GS-15 microtiter tray centrifuge with a collection tray underneath to collect the cleaned up sequencing sample. The collection tray was then placed in a Jouan R6-1010 vacuum centrifuge (Jouan, Winchester, Va.) to dry the samples. The dried samples in the 96 well tray were resuspended in 10 μl each of HI DI formamide (Applied Biosystems, Foster City, Calif.) for loading on the 3100 sequencer. Those dried samples prepared via Centrisep® columns were resuspended in Accutrac™ dye (2 μl for the 377, 3 μl for the 373 sequencer) (Commonwealth BioTechnologies, Richmond, Va.) or in the HIDI formamide for loading on the 3100 sequencer. Once the samples were resuspended in dye they were vortexed and centrifuged at maximum speed for 30 sec. and then heat denatured at 90° C. for 2 min (heat denaturing was only necessary for 373 and 377 samples). The samples were then loaded onto the sequencers. For the 373 and 377 sequencers, Biowhitaker prepackaged Singel® gels (BMA, Rockland, Me.) were used (Long Ranger® Singel® packs for 373 and 377). The gel solution is poured into a side-arm flask for pouring onto the plates, using an Owl Scientific Otter™ sequencing gel caster (Owl Separation Systems, Portsmouth, N.H.) and allowed to polymerize for a minimum of two hours before use. For the 3100, a 50 cm capillary array was used with Pop6 polymer (Applied Biosystems, Foster City, Calif.).

PCR mutagenesis: Random PCR mutagenesis of the pvuIIR gene was carried out using pBBE3 as a template. Four separate 100 μl PCR reactions were carried out with the two primers described above, in a buffer containing 5 mM MgCl₂, 1 mM MnCl₂, and Taq DNA polymerase, in the manufacturer's buffer (New England Biolabs, Beverly, Mass.). Each reaction contained one of the four dNTP's at a concentration of 0.35 mM, with the remaining 3 dNTP's at 0.05 mM. Thirty-five cycles (94° C./30s, 55° C./30s and 72° C./30s) were carried out. The PCR reactions were pooled, purified by adding 2 ml PB buffer, binding to a Qiaprep Spin Column, washing with 0.75 ml PE buffer, and eluting with 50 μl EB buffer (Qiagen, Studio City, Calif.). The resulting DNA was cleaved with BamHI and EcoRI by adding 6 μl 10× NEB EcoRI buffer and 2 μl of each enzyme, then incubating at 37° C. for 16 h (New England Biolabs, Inc., Beverly, Mass.). After adding 7 μl of 10× gel loading buffer, the DNA was run on a 1% low-melt agarose gel (Suprasieve, American Bioanalytical, Natick, Mass.), the DNA fragments were cut out, and treated with 2 units of β-agarase (New England Biolabs, Beverly, Mass.). The DNA was precipitated from the reaction mixture by adding 80 μg glycogen and 2 volumes of ethanol and centrifuging, then resuspending in 20 μl H₂O.

Protein purification: PvuII or its derivative mutants were purified as follows: 10 ml of cells expressing the wild-type or mutant endonuclease were harvested and resuspended in 1 ml of buffer containing 20 mM phosphate (pH 8.0), 300 mM NaCl. These samples were sonicated, followed by centrifugation. The supernatant was collected and mixed with 20 μl of Ni—NTA agarose resin (Qiagen, Studio City, Calif.) for one hour at 4° C. The resin was washed with 20 mM phosphate, 300 mM NaCl, 20 mM imidazole, and the protein was eluted with 20 mM phosphate, 300 mM NaCl, 250 mM imidazole (according to the manufacturer's protocol).

Isolation of K93R, a Relaxed Specificity Mutant of PvuII, and Recycled Mutagenesis to Increase Activity on Non-PvuII Sites

A mutant of PvuII that has activity on sites other than the PvuII cognate site (CAGCTG) was obtained by random PCR mutagenesis of the pvuIIR H84C gene. The H84C mutation had been isolated earlier as having an altered binding specificity, but an unaltered cleavage specificity. After PCR mutagenesis, the pooled PCR fragments were cleaved with BamHI and EcoRI and ligated into pCH1, and the ligation was used to transform PR1553 (which contains the dinD::lacZ allele and the cognate PvuII DNA methyltransferase on pPR1551) to amp resistance. The resulting colonies were screened for blue on LB amp X-gal plates. Candidate blue colonies were grown in LB amp, and plasmid was prepared. Plasmids from the candidates were then sequenced. One mutation that was recovered multiple times was a lysine to arginine mutation in residue 93 of PvuII (K93R), along with the original H84C mutation. Protein was prepared from the double mutant, H84C K93R, and found to have little or no activity in vitro. To investigate the K93 mutation by itself, the single mutant K93R was constructed using the megaprimer method and the primers 5′-GTAATTATTGCAAGATATAGAC AAGTACCTTGGAT-3′ (SEQ ID NO:14) and 5′-TACTTGTCTAT ATCTTGCAATAATTACAGGATTCA-3′ (SEQ ID NO: 15) (Barik, Methods Mol Biol. 192:189-96 (2002)). The pvuIIR K93R PCR fragment was purified using the method described above for random PCR mutagenesis, cleaved with BamHI and EcoRI, gel-purified using the Qiaquick Gel Extraction Kit (Qiagen, Studio City, Calif.), and ligated into pLT7KH cut with BamHI and EcoRI. The ligation was used to transform PR1553 to amp resistance on LB amp plates. Colonies from this transformation became blue when streaked on LB amp X-gal plates containing 100 μM IPTG and incubated for 1-2 days at 30° C., but were very light blue or white when streaked on LB amp X-gal plates containing 10 μM IPTG and incubated similarly.

The K93R mutant protein was purified and its cleavage specificity was examined by cleaving pUC19 DNA. The sizes of the DNA fragments produced showed that it cleaved both unmodified PvuII sites and, at a lower efficiency, sites that differed from the PvuII site at one or two bases, especially CAGGTC.

In order to increase the efficiency of cleavage at the non-PvuII sites, recycled mutagenesis was performed. Random PCR mutagenesis using the pvuIIR K93R gene as template was carried out, and the resulting library of pvuIIR DNA was cleaved with BamHI and EcoRI and ligated into pLT7KH cut with BamHI and EcoRI. The ligation was used to transform PR1553 (dinD::lacZ carrying pPR1551, which expresses the PvuII methylase) to amp resistance, and the transformants were plated on X-gal, amp, cam plates containing 10 μM IPTG, and incubated at 30° C. Under these conditions, PR1553 bearing pLT7KH containing the pvuIIR K93R mutant are white or very light blue. Twenty-eight colonies that were darker blue were used to inoculate 0.2 ml of LB amp cam and grown at 42° C. overnight. Plasmid was purified from these cultures and the DNA was sequenced. The isolates were retested by using their DNA to transform PR1553 to amp resistance, and plating each on two LB amp cam plates, one with 10 μM IPTG, and one with 100 μM IPTG. The expectation is that all the DNA preparations would yield blue colonies on 10 μM IPTG, since these are the conditions of the original screen. DNA preparations that encode mutants of PvuII K93R that have higher activity on sites other than the cognate PvuII site would be expected to have a reduced ability to yield colonies at 100 μM IPTG, and/or would yield colonies that were dark blue. Six of the preparations met these expectations. These preparations were used to transform ER2475 to amp resistance, plated on LB amp cam plates and incubated overnight at 42° C. Fifteen colonies were harvested directly from the transformation plate and resuspended in 10 ml of LB amp cam, grown at 42° C. to an OD₆₀₀ of 0.8-1.0, induced with 1 mM IPTG and incubated for 3 h at 30° C. The cultures were harvested and the mutant endonucleases were purified as described above. Three of these PvuII mutants yielded an appreciable amount of active protein; these mutants were K93R/R105H, K93R/V50A and E18K/K93R/R105H.

Determination of Substrate Specificity

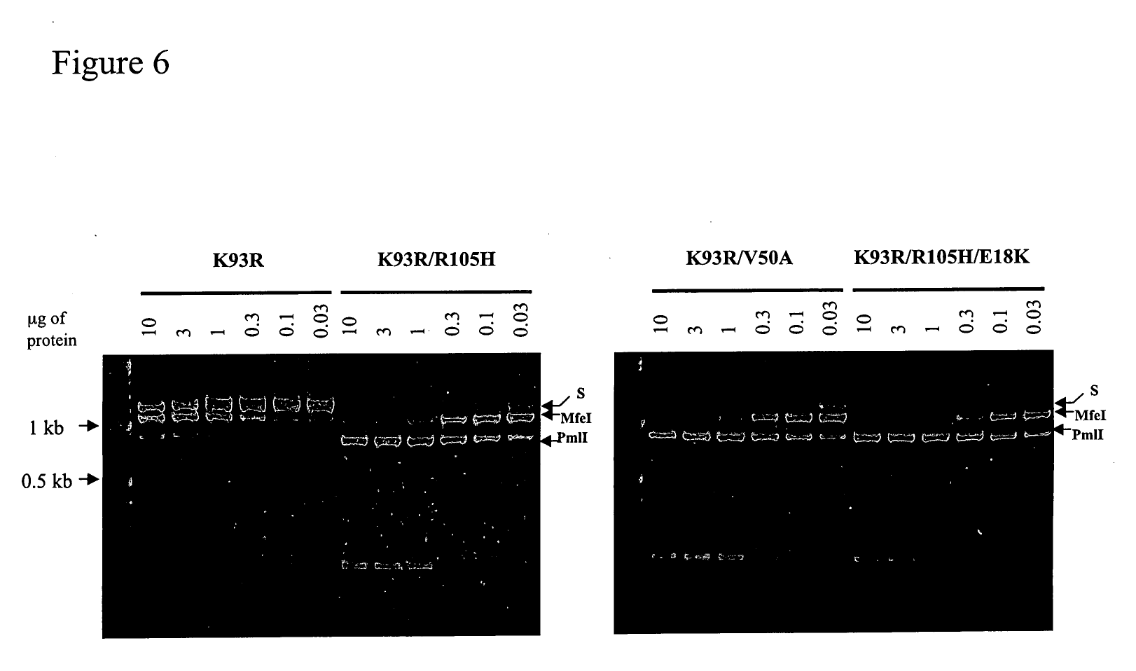

In order to test the cleavage specificity of the mutant endonucleases, two substrates were designed such that a limited number of sites related to the PvuII cognate sequence were present. A 1042 bp DNA substrate, corresponding to bases 317-1359 of the pUC19 sequence (GenBank #L09137), was amplified from pUC19. This substrate contains a unique PvuII site 631 bp from one end. A second, 1270 bp DNA substrate was amplified from the region corresponding to bases 6010-7280 of pTYB1 (New England Biolabs, Beverly, Mass.). This substrate contains a unique MfeI site 1022 bp from one end, and a unique PmlI site 810 bp from the same end. These substrates were used to determine the substrate specificity of PvuII (K93R) and its variants K93R/R105H, K93R/V50A and E18K/K93R/R105H. PvuII restriction digestion was assayed in NEB buffer 2 at 37° C. for 1.5 hour (New England Biolabs, Inc., Beverly, Mass.). As shown in FIG. 6, the amount of protein required to cleave the substrate with the PvuII site for each of the mutants was as follows: 0.03 μg for the K93R mutant, 0.01 μg for K93R/105H, 0.01 μg for K93R/V50A and 0.03 μg for K93R/R105H/E18K. The amount of protein required to cleave the MfeI/PmlI substrate at the MfeI site is >10 μg for the K93R mutant, 0.1 μg for K93R/105H, for 0.1 μg K93R/V50A, and 0.03 μg for K93R/R105H/E18K. The amount of protein required to cleave the MfeI/PmlI substrate at the PmlI site is >10 μg for the K93R mutant, 3 μg for K93R/105H, 3 μg for K93R/V50A, and 3 μg for K93R/R105H/E18K. Cleavage of the MfeI or PmlI sites by the wild-type PvuII endonuclease was not detectible (data not shown). It is clear that, compared to K93R, these K93R variants show an increase in specific cleavage activity at CAATTG and CACTGT, while maintaining similar levels of cleavage activity at CAGCTG.

1. A method of modifying the cleavage activity of a restriction endonuclease comprising the following steps:
(a) creating a pool of mutants of an endonuclease gene coding for a target restriction endonuclease;
(b) transforming a host cell with the mutants of step (a) under conditions where the expressed unmutated gene coding for the target restriction endonuclease generates little or no DNA damage within the host;
(c) screening the pool of transformed mutants within said host cell for an increase in DNA damage; and
(d) obtaining a mutant restriction endonuclease with modified cleavage activity.

2. The method of claim 1, wherein prior to creating said pool of mutants, a predetermined mutation of the restriction endonuclease gene is engineered to alter the specificity of the target restriction endonuclease.

3. The method of claim 2, wherein the altered specificity comprises an altered binding specificity of the target restriction endonuclease.

4. The method of claim 2, wherein the altered specificity comprises an altered cleavage specificity of the target restriction endonuclease.

5. The method of claims 2 to 4, wherein altered specificity is screened for before the screening for increased DNA damage.

6. The method of claims 2 to 4, wherein the altered specificity is screened for after the screening for an increase in DNA damage.

7. BsoBI variants which cleave a subset of the native BsoBI recognition/cleavage site.

8. The BsoBI variants of claim 7, wherein said variants are selected from the group of D246A, D246S, D246T, D246A/C317W, or R38S/L62I/D112Y/N169T/L238F/D246A/A248S.

9. PvuII variants which cleave a subset of the native PvuII recognition/cleavage site.

10. The PvuII variants of claim 9, wherein said variants are selected from the group of K93R/R105H, K93R/V50A and E18K/K93R/R105H.
